(19) United States
(12) Patent Application Publication
(10) Pub. No.: US 2011/0277774 A1
(43) Pub. Date: Nov. 17, 2011

(54) Combination intra oral and extra oral sleep apnea appliance

(76) Inventor: Larry V. Connell, Yakima, WA (US)

(21) Appl. No.: 13/068,047

(22) Filed: Apr. 29, 2011

Related U.S. Application Data
(60) Provisional application No. 61/343,576, filed on Apr. 30, 2010.

(51) Int. Cl. A61F 5/56

(52) U.S. Cl. 128/848

(57) ABSTRACT

A combination intra oral and extra oral sleep apnea appliance, primarily useful as a protector to limit mouth breathing, which generates a noise caused by soft palate vibration while sleeping. The device is therapeutically adapted to be placed within the mouth of a patient to allow free side-to-side movement of the user's lower jaw during use. The device includes upper and lower mandibular members that at least cover the anterior teeth. The upper mandibular member has a tube incorporated into it, running along the lingual incisal edge of the upper teeth. A retaining wire is threaded through this tube and then extend down to attach to the lower mandibular member, which includes two buccal tubes embedded into it to receive the retaining wire. Additionally, a pair of nasal expansion wires can be anchored to a labial tab, mounted externally on the upper mandibular member.

This Non-Provisional Application claims priority to Provisional Patent Application Ser. No. 61/343,576, filed Apr. 30, 2010.

### TECHNICAL FIELD

The invention relates to a therapeutic device adapted to be placed within the mouth of a patient, and more specifically, a combination intra oral and extra oral sleep apnea appliance that is primarily useful as a protector to limit mouth breathing, or breathing through the mouth and nose, which generates a noise caused by soft palate vibration while sleeping.

### BACKGROUND OF THE INVENTION

Obtaining free flowing and adequate oxygen via the breathing air ways is a first priority for any person. The inability of a person to supply adequate oxygen to all the body's cells, in adequate concentration, has become a problem of near epidemic proportions. One in five people suffers from hypoxia and the resulting health problems, which in turn can manifest themselves in many different pathological ways. In as much as all the cells of the body can be affected, any of them may be dysfunctional and display pathology to some degree. However, immediate, and life threatening consequences can stem from the deficient delivery of oxygen to the heart muscles, which can lead to heart failure.

Modern medicine has recognized this issue and has taken steps to find answers as to how to treat these disease manifestations, by devising ways to increase oxygen tension in the blood stream. One such way is to use a Continuous Positive Airway Pressure or CPAP system. This system is presently the highest standard for treating oxygen deprivation, as prescribed after thorough sleep tests, typically performed by a medical sleep specialist, before being recommended. The greatest problem with the CPAP apparatus is patient compliance. Many people find it difficult, for social as well as physical reasons, to wear the CPAP during sleep. Studies have shown that only 40% of those needing the system are able to benefit.

There is a need to simplify the attachment of such devices, like the CPAP, and make the positive air pressure delivery from the system more readily available to the patient. Ideally, an improved device could combine with the CPAP to reduce the amount of air pressure required by some apnea patients. The improved oral appliance could be used as a hybrid with the CPAP, or used alone for patients not needing the pressurized airway system, but only a limited increased opening of the air way, to aid in realizing a benefit of adequate oxygen blood levels.

The following is a disclosure of the present invention that will be understood by reference to the following detailed description taken in conjunction with the accompanying drawings.

### BRIEF DESCRIPTION OF DRAWINGS

FIG. 1 is a perspective view of a combination intra oral and extra oral sleep apnea appliance, according to an embodiment of the invention;

FIG. 2 is a perspective view of a combination intra oral and extra oral sleep apnea appliance, according to an embodiment of the invention;

FIG. 3 is a perspective view of certain elements of a combination intra oral and extra oral sleep apnea appliance, according to an embodiment of the invention;

FIG. 4 is a perspective view of a combination intra oral and extra oral sleep apnea appliance, according to an embodiment of the invention;

FIG. 5 is a top view of a combination intra oral and extra oral sleep apnea appliance, according to an embodiment of the invention;

FIG. 6 is a perspective view of an element of a combination intra oral and extra oral sleep apnea appliance, according to an embodiment of the invention;

FIG. 7 is a perspective view of a combination intra oral and extra oral sleep apnea appliance, according to an embodiment of the invention;

FIG. 8 is a perspective view of a combination intra oral and extra oral sleep apnea appliance, according to an embodiment of the invention;

FIG. 9 is a perspective view of a combination intra oral and extra oral sleep apnea appliance, according to an embodiment of the invention.

FIG. 10 is a perspective view of an element of a combination intra oral and extra oral sleep apnea appliance, according to an embodiment of the invention;

FIG. 11 is a perspective view of a combination intra oral and extra oral sleep apnea appliance, according to an embodiment of the invention;

FIG. 12 is a side view of a combination intra oral and extra oral sleep apnea appliance, according to an embodiment of the invention; and

FIG. 13 is a front view of a combination intra oral and extra oral sleep apnea appliance, according to an embodiment of the invention.

Reference characters included in the above drawings indicate corresponding parts throughout the several views, as discussed herein. The description herein illustrates one preferred embodiment of the invention, in one form, and the description herein is not to be construed as limiting the scope of the invention in any manner. It should be understood that the above listed figures are not necessarily to scale and that the embodiments may be illustrated by fragmentary views, graphic symbols, diagrammatic or schematic representations, and phantom lines. Details that are not necessary for an understanding of the present invention by one skilled in the technology of the invention, or render other details difficult to perceive, may have been omitted.

### DETAILED DESCRIPTION OF SPECIFIC EMBODIMENTS

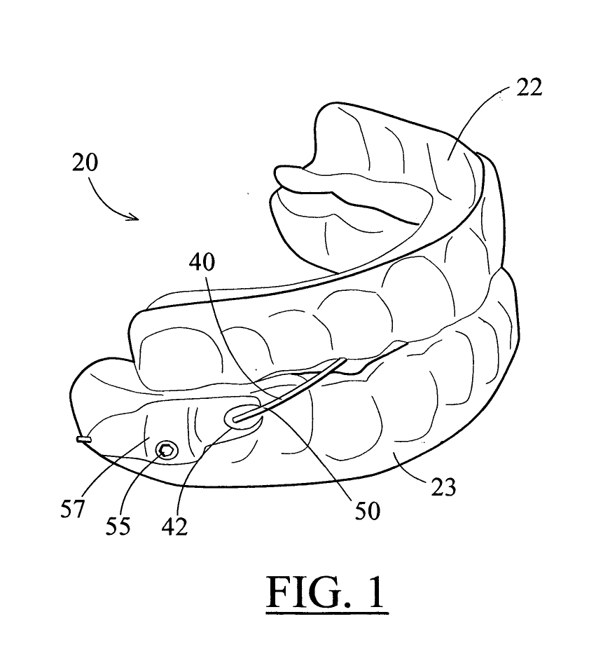

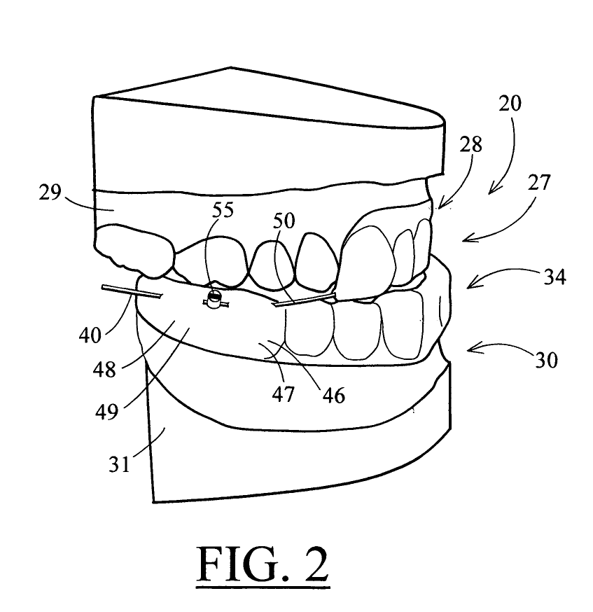

The invention provides a sleep apnea appliance. FIGS. 1 through 13 show the combination intra-oral and extra-oral sleep apnea appliance, according to preferred and alternative embodiments of the present invention. As shown in FIG. 1, the combination intra oral and extra oral sleep apnea appliance 20, simply referred to herein as the “oral appliance,” includes an upper mandibular member 22 and a lower mandibular member 23. The upper mandibular member is a ‘splint’ that at least covers the anterior incisors 25 and canines 26, or upper anterior teeth 27, as shown in FIG. 2. In some cases this splint may cover the entire upper dentition or upper arch 28, of the upper jaw 29, depending on retention factors, such as age and development of the patient, as later discussed herein. The lower mandibular member can also be referred to as a ‘splint,’ and substantially covers the entire lower dentition or lower arch 30, of the lower jaw 31, from molar 32 to molar, which bracket the lower anterior teeth 34. These mandibular member splints are preferably made from durable fracture resistant plastic materials that are well known to dental practitioners in this field of technology, and different patients may require different materials in the construction of the sleep apnea appliance. Some ‘bruxers’ and ‘clenchers’ can cause considerable damage and breakage to the typical appliance made from 0.060 splint plastic. Therefore, more durable materials are preferred for the devices manufactured for such patients.

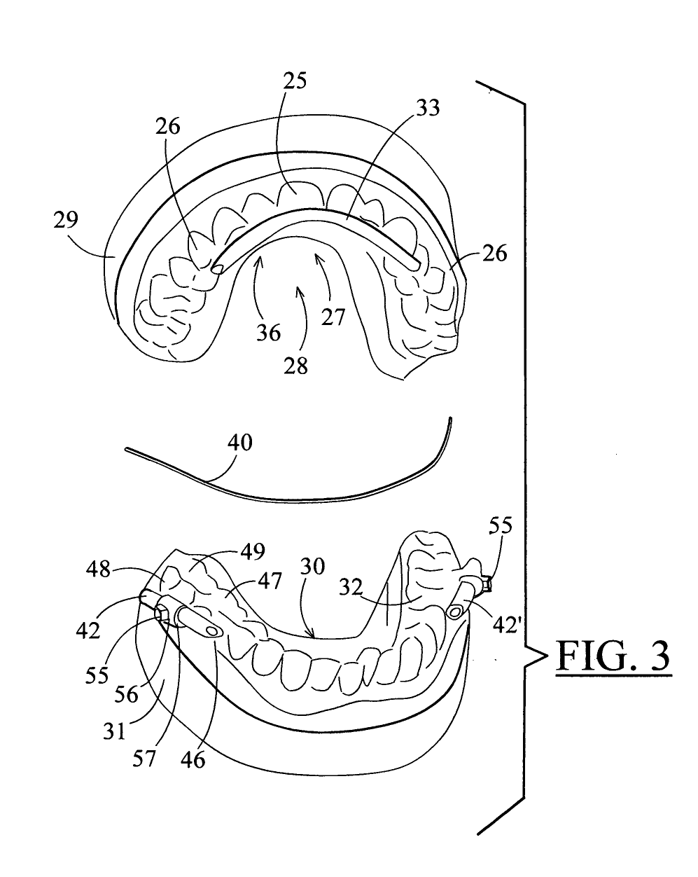

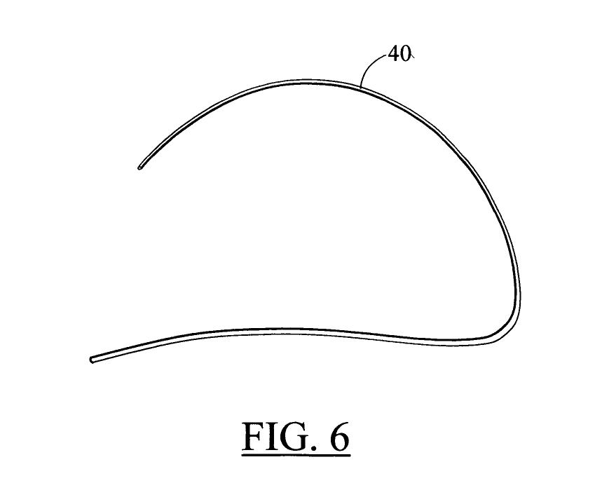

The splint of the upper mandibular member 22 has an upper tube 33 incorporated into it. The upper tube runs, or transverses, from canine 26 to canine along the labial or lingual incisal edge 36 of the upper anterior teeth 27. A retaining wire 40 as shown in FIG. 6, which is preferably a nickel titanium alloy, 0.014 to 0.020 inch in diameter round wire with a high degree of memory retention, is threaded through this tube and then extend down to attach to the lower mandibular member 23. As shown in FIGS. 2 and 3, the lower arch 30 has a pair of, or two buccal tubes 42 and 42′ running from the mesial of the buccal incisal edge 46 of the lower first molar 47 to the distal edge 48 of the second molar 49.

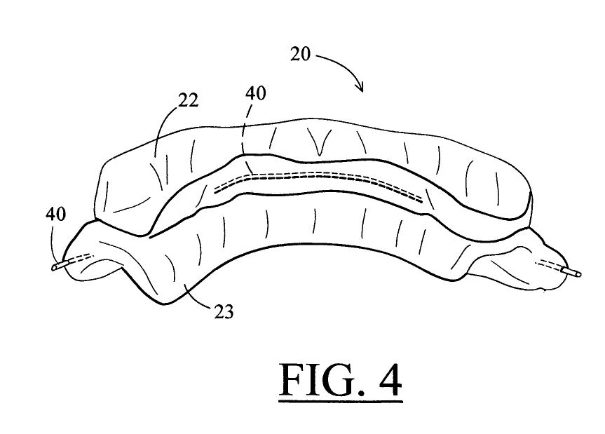

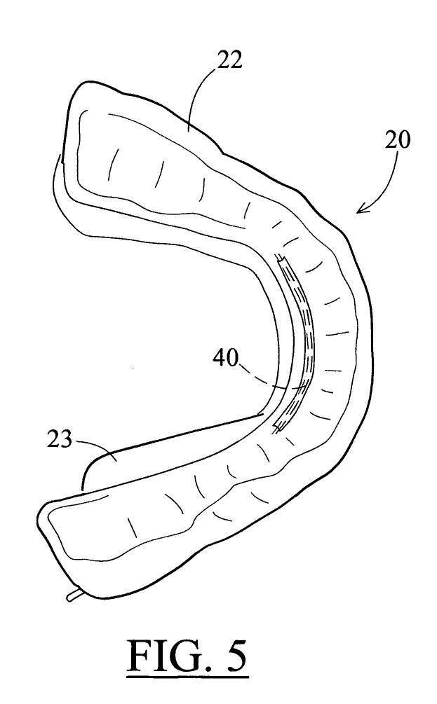

As shown in FIG. 3, the tubing of the buccal tubes 42 and 42′ is embedded into the plastic splint of the lower mandibular member 23, and is placed to receive the retaining wire 40, which has been threaded through the upper tube 33, as shown in FIGS. 4 and 5. This threading allows the splints of the upper mandibular member 22 and lower mandibular member to be interconnected with the flexible and high strength retaining wire. Most preferably, the retaining wire is located in the incisal labial area, for most of its routing through the oral appliance 20. Preferably, the buccal tubes are formed of an appropriate diameter to accommodate the retaining wire 40, and are a USDA approved clear plastic material suitable for internal use, such as an infant gastrointestinal feeding tube, as known by persons skilled in medical tubing technologies. As an alternative, a stainless steel, closed coil spring may be utilized for forming the channel of the retaining wire, instead of the preferred, clear tubing.

Once the lower jaw 31 has been set in a position anterior to the upper arch 28, approximately 5 to 10 millimeters of useable wire of the retaining wire 40, referred to as a wire remainder 50, should still be available, as located near the distal of the upper canine 27 to the mesial of the lower molar 32.

The terms “approximately” or “approximate” are employed herein throughout, including this detailed description and the attached claims, with the understanding that the terms broadly include sizes and measurements that could perform the same purpose or achieve the same results in essentially the same way as the dimensions recited. Additionally, these terms denote a level of exactitude commensurate with the skill and precision typical for this particular field of technology or endeavor, as applicable.

The retaining wire 40 at the wire remainder 50 is then free to slide along the lower two buccal tubes 42 and 42′, as the lower jaw 31 either protrudes or retracts to a final, comfortable position. Every patient using the oral appliance 20 will have a different comfort position however the 5 to 10 mm travel at the wire remainder, usually provides an adequate range to achieve this mandibular position. A set screw 55 is receivable into a set socket 56, which is preferably threaded to allow the set screw to tighten against the retention wire within the buccal tubes. The set socket is preferably a component of a set sleeve 57 that receives one of the buccal tubes, as shown in FIG. 3. The set screw can be tightened at any position to where the patient is comfortable and can tolerate the desired forward position for prolonged periods of time without joint pain. Because the retention wire 40 can slide along the upper tube 33 at the upper labial incisal or lingual incisal edge 36 of the upper anterior teeth 38, the patient has complete freedom to move the lower jaw 29, from side to side, while remaining in the fixed forward position. Also, the angles of the upper tubing and lower two buccal tubes 42 and 42′ can vary to compensate for the many different kinds of jaw relations encountered in the general population. For example, in a conventionally defined Class I, II, or III occlusions, a dental practitioner is able to customize the manufacture of the oral appliance 20 of the present invention, to harmonize the upper arch 28 with the lower arch 30, and to keep within the ‘freeway space’ of the patient. This feature allows the lips 60 of the patient to remain near the closed position during the night, to avoid excessive dry-mouth and to encourage nasal breathing.

Preferably, the upper splint oral appliance 20 includes an upper flange 62 for covering the buccal bone, as shown in FIG. 2. This upper flange is beneficial to increase the anchorage of the upper mandibular member 22, when the lower arch 30, in its forward position, creates a distal force on the upper anterior teeth 27. Otherwise, this excess distal force can loosen the six upper anterior teeth.

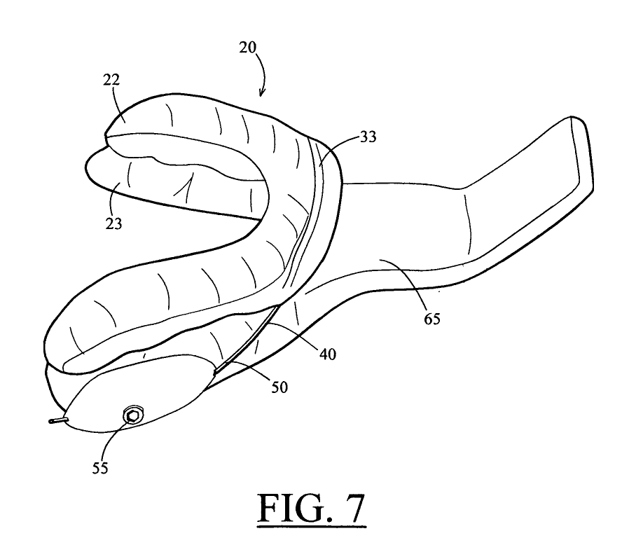

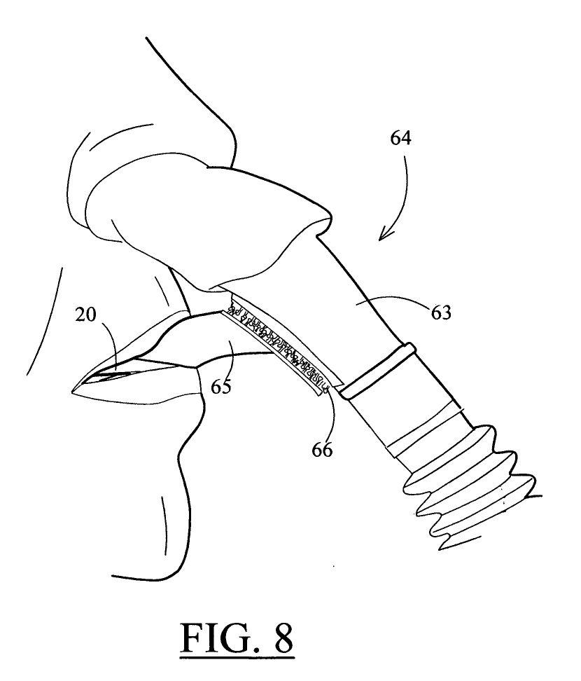

As shown in FIG. 8, the oral appliance 20 can be modified to attach to a nose tube 63 of a conventional CPAP (Continuous positive air pressure) appliance 64. The nose tube of the CPAP appliance can be mounted to the oral appliance 20 at a tab extension 65, as shown in FIG. 7. The tab extension preferably extends from the lower mandibular member 23, proximate to the attachment 66 of the oral appliance, which may be achieved with a ‘velcro’ type of connection, as also shown in FIG. 8. This alternative embodiment provides a secure attachment that eliminates head straps and movement of the CPAP nose tube during sleep.

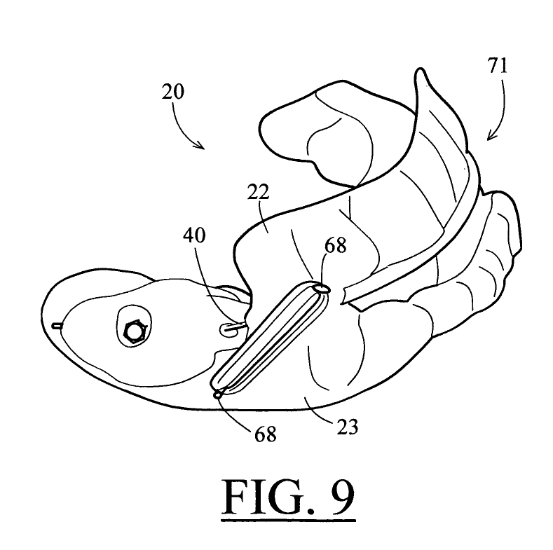

Further enhancements of the oral appliance 20 can include a button attachment 68 on the upper mandibular member 22 and lower mandibular member 23, for up and down elastics 70 to be used to help secure the oral appliance in a closed position 71, as shown in FIG. 9. Also, alternatively, the retention wire 40 can be a reverse vector retraction 0.017 by 0.025 inch rectangular in cross section of the preferred nickel-titanium alloy, and can be inserted in place of the preferred round wire to provide additional strength and force to keep the user's mouth closed during sleep. Again, different patients may require differently shaped, or heavier gauge materials in the construction of the retention wire, up to or greater than approximately 0.10 inch diameter.

The oral appliance 20 of the present invention is a ‘tooth anchored’ apnea appliances that can serve the two classes of such intra-oral appliances:

A ‘Class I’ appliance, which is employed for treatment of mild apnea cases with the standard AHI of 10 to 15 and below, and an oxygen saturation within the 90th percentile, and where the CPAP appliance 64 has proven not usable by the patient. The oral appliance 20 of the present invention can be used as a Class I appliance, and is completely tooth-born and invisible, when worn by the patient.

A ‘Class II’ appliance has only the upper arch 28 of teeth, serving as an anchor that is in turn attached to the pressurized nose tube 63 of the CPAP appliance 64, which is received into the nose 67 of the patient, as shown in FIG. 8. The oral appliance 20 of the present invention totally eliminates the usual head straps required to hold the nose tube in place.

When used as a Class I appliance, the oral appliance 20 can also utilize the lower mandibular member 23 to bring the lower jaw 31 forward, as well as accommodate the pressurized CPAP nose tube 63. This combination of the Class I and Class II features can facilitate keeping the mouth closed during the sleep period as well as reduce the CPAP appliance 64 delivery pressure necessary to achieve adequate oxygen blood levels. The oral appliance can also be used to relieve stress to the ‘temporal mandibular joint’ (TMJ) during the sleep period. This appliance combination can be referred to as a ‘Class II Hybrid.’

As a Class I appliance, the oral appliance 20 of the present invention can function as a therapeutic mandibular advancement device, with the following advantages, unique improvements and multiple functional purposes:

The oral appliance 20 provides for incremental advancement of the lower arch 30 within the freeway breathing pathway of the patient, thereby reducing stress on the TMJ, and also allows mouth closure, while promoting nose breathing.

Most preferably, the upper mandibular member 22 and lower mandibular member 23 of the oral appliance 20 optionally cover all, or just the six anterior maxillary teeth, which leaves the palate free for more patient comfort. In general the oral appliance has far less tooth and soft tissue coverage providing superior patient comfort and acceptance with hours of use.

As shown in FIG. 8, the oral appliance 20 can be attached to the CPAP to reduce the amount of air pressure required to maintain an open airway for more severe apnea patients. This advantage is realized by bringing the lower jaw 31 forward thereby opening the air way that would otherwise need to be opened by increased air pressure with the CPAP. With the ability to reposition the retention wire 40, the oral appliance has total adjustability in both the Class I and Class II applications. As a Class I or Class II appliance, it can be modified and attached to any existing CPAP system presently on the market.

The oral appliance 20 allows free movement of the user's mandible or lower jaw 31, from side to side during use of the appliance, so as not to hold the patient in one position for prolonged periods of time. This reduces stress on both the muscles of mastication and the ligaments associated with the TMJ. Furthermore, the oral appliance can simultaneously reduce TMJ stress by bringing the joint forward and out of the trauma relationship. Care must be used, if the condyle is too far forward and has slipped of the joint disc, as commonly referred to as a ‘reciprocal click.’ This is a contraindication for the use of the oral appliance and may worsen the internal joint pathology. The meniscus needs to remain between the condyle and the fossa during all jaw movements.

By being functional within the patient free way space the oral appliance 20 reduces mouth breathing and subsequent drying during the sleep interval. Up and down elastics between the upper mandibular member 22 and lower mandibular member 23 can insure mouth closure if necessary. In brief the appliance is functional while still allowing the patient to remain within the normal freeway space. Studies show that nose breathing versus mouth breathing can increase oxygen uptake in the blood stream by up to 25%.

The oral appliance 20 can be custom made to fit patients with deep Class II, division 1 and division 2 malocclusions, or with Class H and Class I occlusions, by altering the angle of the tubing on both the upper tube 33 in the upper mandibular member 22, receiving the close coil spring of the retaining wire 40, and the lower buccal tubes 42 and 42′ in the lower mandibular member 23. This adjustability adds significantly to the comfort, function and long term use of the appliance.

Because the oral appliance 20 has infinite adjustability in the anterior-posterior directions, the appliance can be used on most patients exhibiting TMJ joint dysfunction, reducing stress on the enervated portion of the meniscus in the glenoid fossa. Additionally, the oral appliance can be used to recapture the meniscus or disc, in the event it is locked out of the fossa into a forward position in relation to the articular eminence. This therapeutic procedure employing the oral appliance can facilitate patent comfort and better patient compliance, while simultaneously treating the apnea.

In another use of the oral appliance 20, it can be utilized as an ‘anti-bruxing’ device, when the lower arch 30 is advanced forward to where only the incisal teeth or the upper arch 28 and the lower arch 30 can come into contact, at which point it is impossible to clench the molars 32. This can reduce tension on the muscles of mastication and surrounding head and neck muscles and in turn provide better airway patency. The oral appliance can also be reinforced on the lower posterior occlusion with high resistance fracture plastics to resist breakage, surrounding the set socket 56 and set sleeve 57, which each receive one of the lower buccal tubes 42 or 42′, as shown in FIG. 3.

One advantage of the oral appliance 20 as compared to conventionally available devices is that it allows near normal speech by the user. However, even though the patient can speak with the oral appliance inserted, they will find it impossible to eat. For this reason, the oral appliance could be worn full time and used as an aid in curbing calorie intake, to help patient lose weight, which may also aid in reducing airway obstruction.

The use of the set screw 55 to control lateral expansion of the retaining wire 40 in the upper tube 33 of the oral appliance 20 can accomplish an expansion of the upper arch 28, which will subsequently expand the nasal airway, and open up the upper arch so the lower arch 30 can be less restricted and allow the condyle to move forward in the fossa to relieve internal joint pressure. Both airway and TMJ function can be improved. As a Class I appliance, the oral appliance has been found to be very effective in eliminating benign snoring, and the appliance can be adapted to children with compromised airways.

To adjust the tension between the upper mandibular member 22 and the lower mandibular member 23 the oral appliance 20, selectively alternative wire diameters can be employed to connect the upper mandibular member to the lower mandibular member. For example, a 0.014 round nickel titanium retention wire 40 could replace an originally installed 0.020 wire. Also rectangular reverse vector retraction wire nickel-titanium 0.017×0.025 inch wire can be inserted alone or in combination with a round wire to increase mouth closure tension. The length of the slack wire from the exit from the upper closed coil spring to the entrance into the lower fixed tube could greatly increase the flexibility and movement of the lower jaw 31.

In an additional alternative embodiment of the present invention, a heat sensitive electronic sensor can be incorporated into oral appliance 20 to measure the patients body temperature, verifying that the oral appliance is fully seated. Using a wireless transmitter or ‘RFID chip,’ this information can be recorded in terms of time and temperature on a nearby receiver, providing a record and verification of how long the patient wore the oral appliance during any given sleep period. This information along with follow-up sleep study can then verify appliance efficacy and satisfy legal requirements for people needing to show eligibility to function in certain jobs requiring a level of normal sleep. One example is a truck driver needing to hold a commercial license. Current laws require him to take sleep studies and show reasonable treatment, if apnea is present, in order to maintain legal driving status.

The oral appliance 20 can be attached to an upper denture, and therefore able to accommodate edentulous patients. Additionally, it can be used in place of jaw advancement or telegnathic surgical therapy for the treatment of sleep apnea. The lower jaw 31 can be brought forward to a known comfortable position to where the airway has been expanded and the occlusion approached a class one relation. In the case of a Class II subdivision 2, the upper anterior teeth 27 are moved forward which also provides space for the lowers to also be moved forward. This is followed by using up and down elastics to bring the remaining occlusion into a Class I relationship. Thus, the lower jaw is moved to a more esthetic position as well as functional, as disclosed in the American Association of Oral and Maxillo Facial Surgeons Update, Vol 22, issue 1. This procedure has the high success rate in correcting Sleep Apnea of 95 to 100%.

The oral appliance 20 may be difficult to use in treating children because of the dynamic transition of the dentition from the primary to the permanent teeth. A dental practitioner cannot interfere on any long term basis with the erupting of permanent teeth. Also, appliance retention is typically difficult on the short primary and newly erupted permanent teeth. Considering the crowding of the dentition and breathing problems often found in conjunction with airway compromised children in these cases with mild to moderate apnea, an alternative embodiment of the oral appliance can include a changeable combination of airway and orthodontic therapies.

For a first stage, a base splint, which is essentially the upper mandibular member 22 and the lower mandibular member 23 of the oral appliance 20, is fabricated. Preferred forms of the upper mandibular member and lower mandibular member for this embodiment are shown in FIG. 2. These base elements can receive the retaining wire 40 for retention and expansion of the upper arch 28 and lower arch 30, to provide room for the erupting teeth, breaking of the palatal bony suture that will intern expand the nasal cavity and increase nasal airway. Finally it will hold the six year molars in place and preserve the leeway space to add another three-millimeter space for the crowded dentition in each quadrant.

In a second stage, the lower buccal tubes 42 and 42′ and set screws 55 are added to the lower mandibular member 23 of the oral appliance 20. The upper tube 33 is added along the upper lingual incisal edge 36 of the upper mandibular member 22, as previously described in this patent to receive the nickel-titanium retaining wire 40. The retaining wire can be set each night to move the patient's jaw forward to accomplish the opening of the airway. For this embodiment, during the day the wire is removed and the patient can continue wearing the upper mandibular member and the lower mandibular member, to accomplish the orthodontic desired results. When the orthodontics is completed and the permanent teeth have erupted and the arch is expanded, the splints of the appliance are converted into retainers, with the upper mandibular member and the lower mandibular member continuing to serve as a pull forward appliance during the sleep period.

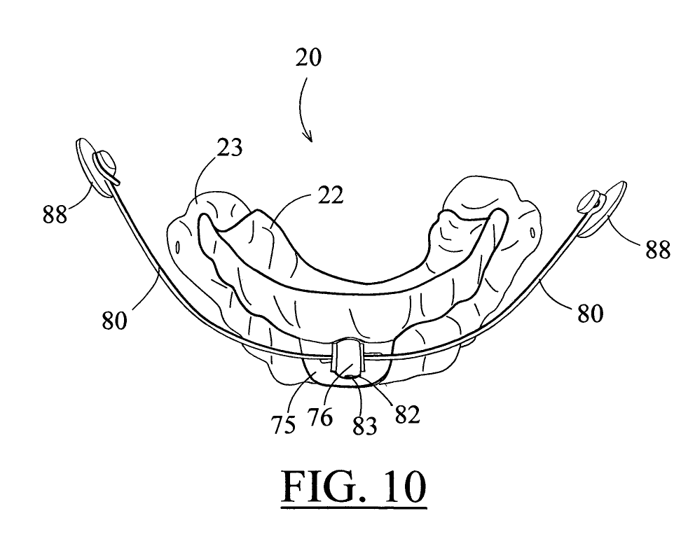

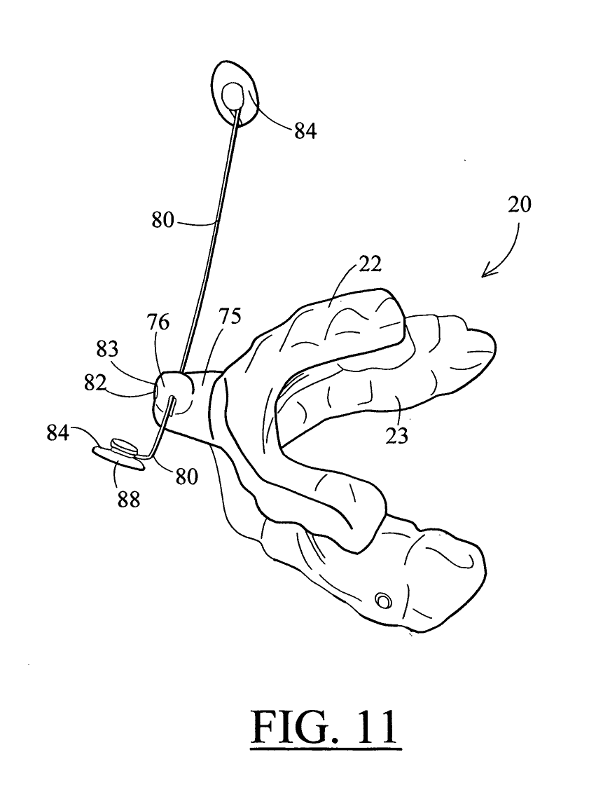

In an additional embodiment of the oral appliance 20, a nasal passage expander 70 can be included, which provides a nasal passage expansion benefits. A pair of nasal expansion wires 80 can be anchored to a labial tab 75, which mounts externally, proximate to the upper anterior teeth 27 on the upper mandibular member, as shown in FIGS. 10 and 11. The labial tab is similar to the tab extension 65, as optionally mounted proximate to the lower anterior teeth. The labial tab includes a nasal set sleeve 76, which receives the pair of nasal expansion wires in each end, as shown in FIG. 10. Employing a nasal set-screw 82, received into a nasal set-socket 83 located on the nasal set sleeve as shown in FIG. 10, one of the pair of nasal expansion wires are received and secured into each end of the nasal set socket, held in place with the nasal set-screw.

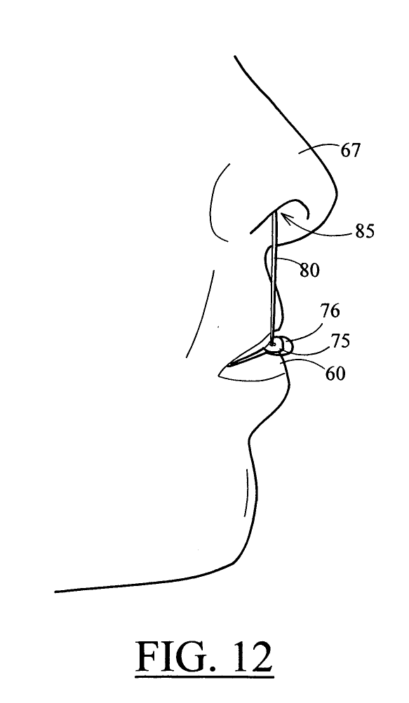

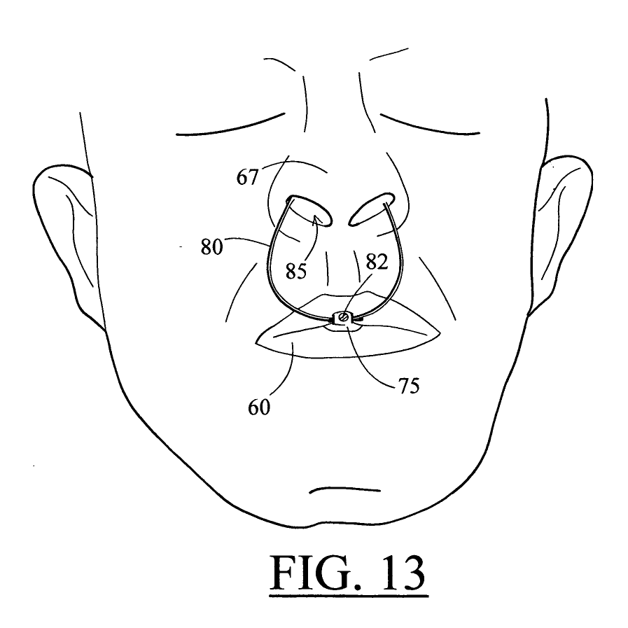

The pair of nasal expansion wires 80 are preferably of a similar diameter and composition as the retaining wire 40, discussed herein above, with the nasal expansion wires also able to be adjusted in length, shape and diameter. Preferably, nare buttons 84 are placed at the distal ends of the nasal expansion wires, as shown in FIGS. 10 and 11. The nare buttons are adapted for receipt into the internal nares 85 within the nose 67 of the patient, and can include pads 88 for cushioning or receiving medications. The medicated pads on the nare buttons can provide any needed medications to add to the comfort of the nare buttons, or additionally administer any need medications during the use of the nasal expansion features. With nasal expansion wires coupled with the nare buttons, pressure can be directed and placed to expand the nostrils to any desired comfortable position, as shown in FIGS. 12 and 13.

The use of the oral appliance 20 with the nasal expansion option can increase the volume of air that can be inhaled during the sleep period and improve blood oxygen tension as a result. The nasal expanders are especially helpful in UARS (upper airway respiratory syndrome) cases, where the CPAP appliance 64 cannot be used. The pads of internal nares buttons can be impregnated with topical medications, such as those suited to relieve congestion during sleep. Alternatively, pad sleeves can be placed over the nare buttons, to contain and administer the desired medications.

In compliance with the statutes, the invention has been described in language more or less specific as to structural features and process steps. While this invention is susceptible to embodiment in different forms, the specification illustrates preferred embodiments of the invention with the understanding that the present disclosure is to be considered an exemplification of the principles of the invention, and the disclosure is not intended to limit the invention to the particular embodiments described. Those with ordinary skill in the art will appreciate that other embodiments and variations of the invention are possible, which employ the same inventive concepts as described above. Therefore, the invention is not to be limited except by the following claims, as appropriately interpreted in accordance with the doctrine of equivalents.

The following is claimed:

1. A sleep apnea appliance comprising:
an upper mandibular member covering a user's anterior teeth, and a lower mandibular member substantially covering the user's lower arch of teeth, and the lower mandibular member having a first buccal tube and a second buccal tube, the first buccal tube and a second buccal tube embedded within the lower mandibular member;
a tube incorporated into the upper mandibular member, the tube positioned along an incisal edge of the user's upper teeth;
a retaining wire the first buccal tube receives the retaining wire, the retaining wire threaded from the first buccal tube through the tube, to the second buccal tube; and
the wire attached to the lower mandibular member at the first buccal tube, and the wire attached to the lower mandibular member at the second buccal tube.

2. The sleep apnea appliance of claim 1, wherein:
the upper mandibular member additionally covers an entire upper dentition of the user's upper jaw.

3. The sleep apnea appliance of claim 1, further comprising:
a pair of nasal expansion wires anchored to a labial tab, the labial tab externally mounted on the upper mandibular member, and each of the pair of nasal expansion wires for insertion into a nostril of the user.

4. The sleep apnea appliance of claim 1, wherein the retaining wire is alternatively removable and re-mountable.

5. The sleep apnea appliance of claim 1, wherein, the tube positioned along the incisal edge of the user's upper teeth is positioned along an incisal edge of the user's upper teeth.

6. The sleep apnea appliance of claim 1, wherein, the tube positioned along the incisal edge of the user's upper teeth is positioned along a labial edge of the user's upper teeth.

7. A sleep apnea appliance comprising:
an upper mandibular member and a lower mandibular member, the upper mandibular member covers a user's upper anterior teeth, and the lower mandibular covers the user's lower arch of teeth;
an upper tube incorporated into the upper mandibular member, the upper tube transverses an incisal edge of the user's upper anterior teeth;
a retaining wire threaded through the upper tube and retaining wire extend to attach to the lower mandibular member; and
a pair of buccal tubes embedded into the lower mandibular member, with each of the pair of buccal tubes placed to receive the retaining wire, with the upper mandibular member and lower mandibular member interconnected with the retaining wire.

8. The sleep apnea appliance of claim 7, wherein:
the upper mandibular member additionally covers an entire upper dentition of the user's upper jaw.

9. The sleep apnea appliance of claim 7, further comprising:
a pair of nasal expansion wires anchored to a labial tab, the labial tab externally mounted on the upper mandibular member, and each of the pair of nasal expansion wires for insertion into a nostril of the user.

10. The sleep apnea appliance of claim 7, wherein the retaining wire is alternatively removable and re-mountable.

11. The sleep apnea appliance of claim 7, wherein, the tube positioned along the incisal edge of the users' upper teeth is positioned along an incisal edge of the user's upper teeth.

12. The sleep apnea appliance of claim 7, wherein, the tube positioned along the incisal edge of the user's upper teeth is positioned along a labial edge of the user's upper teeth.
